(19) United States
(12) Patent Application Publication
(10) Pub. No.: US 2021/0178004 A1
(43) Pub. Date: Jun. 17, 2021

(54) Multi-chambered and extended release fragrance and scenting devices

(71) Applicants: Sven Dobler, Huntington, NY (US); Russell Ostroff, West Islip, NY (US); Varleriano Marinelli, Port Washington, NY (US); Per Dobler, City of Huntington, NY (US)

(72) Inventors: Sven Dobler, Huntington, NY (US); Russell Ostroff, West Islip, NY (US); Varleriano Marinelli, Port Washington, NY (US); Per Dobler, City of Huntington, NY (US)

(21) Appl. No.: 16/974,286

(22) Filed: Dec. 17, 2020

Related U.S. Application Data
(60) Provisional application No. 62/974,687, filed on Dec. 17, 2019.

(51) Int. Cl. A61L 9/04; A61L 9/12

(52) U.S. Cl. CPC A61L 9/048; A61L 9/12

(57) ABSTRACT

This invention is an air freshener, one that can be applied to consecutively disseminate scented material into the air, and includes a base member, the base member having a plurality of chambers or cavities provided therein, and into which a fragrance emitting gel may be deposited, during manufacture, with each cavity having a seal member applied thereon, to seal in the gel material. Then, the seal members may be removed consecutively, or in certain instances simultaneously, in order to disseminate the air freshening material from the contained gel to provide for the admittance of fragrance aroma into the surrounding atmosphere. In another embodiment, the base member may have a series of laminates applied to it, each laminate having a fragrance emitting material applied thereto, so that as each laminate is removed, it provides for a sustained emission of a fragrance aroma into the surrounding atmosphere, to function as an air freshener. The base members may be suspended by a cord from a support, whether it be in the vehicle, an office, or a room in the home.

### CROSS REFERENCE TO RELATED APPLICATIONS

This is a regular letters patent application upon the provisional application having Ser. No. 62/974,687, filed on Dec. 17, 2019, and this application is a continuation-in-part of the patent application having Ser. No. 15/732,156, filed on Sep. 26, 2017, and which latter application is the non-provisional of its provisional patent application having Ser. No. 62/496,240, filed on Oct. 7, 2016.

### FIELD OF THE DISCLOSURE

This disclosure generally relates to an air freshener, and more particularly to an air freshener that provides for the multi-chambered holding of fragrance material, providing for its consecutive, or even simultaneous release at the selection of its user, of its fragrance to the ambient atmosphere.

### BACKGROUND OF THE INVENTION

Air fresheners for automobiles typically consist of a printed paper card that is dosed with fragrance and provided with a string for hanging from a rearview mirror or other structure within the automobile or other vehicle. The air freshener for the home may undertake any type of a wicking device, that is immersed in the liquid fragrant material, and once opened, provides for the dissemination of the fragrant aroma into the ambient air. Or, there are a variety of sprays that are used to refreshen the air of a room, as known in the art. Usually, in the individual air freshener type of device, it is wrapped in an odor barrier containment package until it is to be used. The air freshener is also attached to a header card for display of the air freshener, at the retail location.

Most air fresheners work by dosing a liquid fragrance formulation into an absorbent material, and then allowing the fragrance to be released during usage, by evaporation, to the surrounding environment over time, but once the freshener is opened, it usually remains as such, until its aroma is depleted. The rate of evaporation is a critical factor of functionality. A quick evaporation of the fragrance usually renders the air freshener overpowering upon removal of the wrapper, and results in a very short usage period. To the consumer, this experience can be unsatisfactory. The consumer may experience too much fragrance immediately, which can be unpleasant and even irritating to the eyes as shown. Further, if the air freshener evaporates rapidly, then the consumer will have to repurchase a new freshener, within a few days, since the air freshener will have dried out and there is little if any fragrance left to be released. It is also possible that the fragrance will evaporate too slowly. If this occurs, then there will be an insufficient impact to mask the odors or even be noticed by the consumer. This also can be unsatisfactory to the purchaser. Since too little fragrance is evaporating on a daily basis, there will be no initial impact by using the device, and no sustained impact over time.

The rate of evaporation of the air freshener is a function of many interrelated factors. The thickness of the absorbent material used for the printed card or substrate is a factor. For example, depending upon the material that is used, the more liquid the material used, the faster the fragrance can be released. The thicker the material that is used, the slower the fragrance will be released into the atmosphere. Another factor deals with the nature of the absorbent material or fragrance holder. The use of paper, plastic, paperboard, and wood are common as a fragrance supporting substrate, but liquid carriers such as pumps, bottles combined with wicks or paper, as stated, and liquid membrane containers, can also be used. The placement of the air freshener is a factor. In particular, the air freshener may suspend from a rearview mirror, or be placed under a sink, positioned in or at an air vent, sprayed actively on demand or passively evaporated. The surface area of the air freshener also is a factor. The larger the surface area of the air freshener the more fragrance it can hold, and the quicker the fragrance will be released into the air, and the smaller the surface area of the air freshener the slower the fragrance will be released. Another important design consideration is the fragrance formulation. A perfumer can increase or decrease the volatility of a fragrance formulation to control the rate of evaporation. Fragrance load can also be adjusted to control the rate of evaporation of the fragrance. Specifically, the higher the fragrance load into the absorbent material or container the more power and longevity the air freshener can be expected to exhibit during usage. The overall size and surface area of the air freshener is a factor that can be altered to control the rate of evaporation of the fragrance. The smaller the carrier or surface area, the less fragrance that the carrier can hold and release. The release of the fragrance may be for a small period of time due to the size of the carrier, and the impact of the fragrance may be weak.

Conversely, a large surface area will allow rapid and increased release of the fragrance, rendering the air freshener stronger. If the fragrance load is not also increased, the air freshener will soon have lost its potency.

Ambient temperature also has an impact on the rate of evaporation. For example, the higher the ambient temperature the quicker the fragrance will evaporate. Also, the humidity of the environment in which the air freshener is placed must be considered. In dry air, the air freshener will release fragrance more rapidly than in humid air. Finally, the air flow surrounding the air freshener is another factor that affects the rate of evaporation. The higher the air flow surrounding the air freshener, the faster fragrance will be released.

Manufacturers and marketers of air fresheners have attempted to balance the above enumerated factors and constraints when designing air fresheners, and in certain places and during climatic seasons the use of one type of air freshener may be preferred to that of another air freshener. When combined with consumer aesthetics and use preferences, a myriad of choices of air fresheners area available. For example, air fresheners such as paper, plastic, pumps, liquid membrane, vent clip auto, hanging, under seat, and tabletop are available for selection and purchase.

When designing air fresheners there are limitations in manufacturing thicknesses and characteristics of absorbent papers. Fourdrinier type paper machines can produce papers with smooth surfaces which is desirable for print decoration, but can generally produce a maximum thickness of only 30 point. In order to increase the thickness that is necessary to use another paper ply that is glued to the 30 point thick ply, as a lamination, to build up the thicknesses to an industrial standard of 60 point. Multi-ply papers that use an adhesive are more prone to separation as the fragrance materials can dissolve the adhesive, as can be understood. Cylinder board machines can be used to produce thicker papers, but many of these papers are not suitable for printing and decorating due to their thickness, and rougher surface properties. Plastics have limitations relative to thickness in the molding process as well. Hence, as can be understood, it is very difficult to control the release of fragrances from their holders, and it is even more difficult to attain an air freshener that can be timed, in its usage, in Order to sustain the release of the air freshener, into the atmosphere, over an extended period of time.

Size surface area for paper air fresheners is often constrained by three main factors. These three main factors are the width of the standard peg location at the retail location, and the concern to avoid obstruction of a driver's view during usage, and the need for displaying marketing and branding information as well as the fragrance load. Generally, a paper air freshener has a thickness between 55 point and 80 point, but thinner and thicker air fresheners can also be found. Nevertheless, in their usage, they are usually a one time application, and will release and dissipate its fragrance, without any further control. The paper air freshener may have printing or decoration that is applied by use of lithographic offset or silk screen for thicker paper. The air freshener has a maximum size of generally 2.5 inches by 5 inches. The fragrance load will vary between one gram and two grams. That is the one time load, that once the fragrance material is exposed, through removal of its wrapper, the fragrance load will dissipate continuously. However, the thicker air freshener has problems such as being prone to separation and difficult to decorate. Also, increasing the size of the air freshener may prevent it from being displayed on a peg rack, and decreasing the size will reduce the ability to load sufficient fragrance into it. In view of this, it should be appreciated that an air freshener manufacturer is faced with many limitations in adjusting the various factors to create a long lasting air freshener having a steady, continuous, and linear evaporation profile.

There are a variety of air freshener dispenser devices, such as can be seen in the United States Patent to Martin, et al., U.S. Pat. No. 5,788,155. This shows an air freshener dispenser having a single shallow tray with a volatile air freshener, and an outer peelable shield to cover the tray before usage.

The published application to Rome, U.S. 2015/0014429, shows a portable type device and method for providing scents. This is a pen like device holding cartridges of scents, and which can be opened for release of scents.

These are examples of some fragrance releasing devices that furnish the dissemination of their scents.

The concept of this current invention takes all of these prior art devices much further, by providing a multi-chambered and continuous release of a fragrance and scenting device, wherein fragrances may be released in step fashion for an extended period of time, and, for example, one part can release its fragrance during a first day or week, and then other parts that can be staggered in their opening for releasing their fragrances over additional periods of time, to furnish a long term air freshening, through the use of a singular device.

### SUMMARY OF THE DISCLOSURE

The concept of this invention is to build upon and augment the type of air freshener as shown in our previous United States patent application having Ser. No. 15/732,156, the teachings of which are incorporated herein by reference. In that application, we showed a form of air freshener that comprises a body, having a vessel like appearance, and which body had an amount of fragrance loaded therein, and then a barrier sleeve positioned over the fragrance saturated body portion, and which sleeve could be either removed or repositioned in order to allow for the gradual release and dissemination of its fragrance into the surrounding atmosphere.

The concept of this current invention takes that freshener a step further, and provides the means for furnishing a controlled release, over an extended period of time, of the fragrance material from its body portion or holder, so that the freshener can operate to release its fragrance into the ambient air, over a regulated period of time, which may be for days, or even weeks, or for a month, depending upon its method of usage and application.

Furthermore, the concept of this invention is generally embodied in three types of components, that allow for the controlled release of fragrance, which initially, includes a saturated paper stock, or a polymer like tray that has been molded having a series of chambers formed therein, whether it be formed by indentation, or molded in situ, in the case of a plastic or metal tray, with a series of seals provided upon the back or front surface of the tray, and which can be sequentially removed, to attain a dissemination of its held fragrance gel, into the atmosphere, over a sustained period of time, and when that particular segment of the device has been depleted, a second or additional seals may be removed, for continuing the dissemination process of its fragrance material to the ambient air, for a very sustained and timed release of the fragrances to the ambient atmosphere. Thus, for example, once the seal has been opened for one of the chambers of the multi-chambered and staggered release of fragrance and scenting device, sometime later, after the initial fragrance or its gel has been depleted, a second or additional seals may be opened, to allow for the continued use of the scenting device, over a sustained and long period of time. Thus, each chambered fragrance will be released, only after its particular seal has been broken, or opened, which may be consecutive, sequentially, or done at different times, depending upon the need and necessity for cleansing the ambient air, and masking it with a scented aroma, as released from the use of this device.

A second embodiment of this invention may include what is identified as a hanging and adjustable type of slider form of fragrance delivering device, and this will include, once again, a formed tray, having at least one chamber provided therein, holding a quantity of the fragrance material or fragrance gel, while the cover for the formed chamber, holding such material, can be slid in a direction to gradually open the fragrance device, with generally the cover being slid downwardly, in stepped degrees, to gradually release the aroma from the scenting material, to the atmosphere, during the sustained usage of this particular slider type of air freshener. Obviously, the sliding cover could be closed, at any time, when the user does not desire to add any additional scented material into the atmosphere, or the cover may be slid downwardly, to expose additional quantities of the fragrance material, over a prolonged period of time, when used for scenting purposes. For example, this type of sliding fragrance disseminating device may be appropriately used in the automobile, such as commonly done, by suspending it from the support for the rearview mirror, in preparation for its application and usage.

A third embodiment that provides for the sequential delivery of fragrance material into the surrounding atmosphere, is formed, once again, from a paper or polymer type of body, and which has one or more layers of the scent delivering sheets, laminated upon its surface, and when each sheet is removed, one from upon another, additional fragrance may be added to the atmosphere, to act as an air freshener. For example, as one well knows, there are the perfumed sampling devices that are appended to the pages, for example, of a fashion magazine, and when the sheet is pulled open, a perfume aroma is released, for sampling by the reader of the magazine. This aspect of the current invention, is to laminate more than one of these sheets onto the surface of a body, of the type that may be suspended from a rearview mirror of a vehicle, or elsewhere, and usually after a cover is removed, the top layer sheet may be removed, for disseminating its freshener into the atmosphere, and when that becomes depleted, after some time, the second layer may be removed, to release further fragrance into the atmosphere, or to initiate another level of air freshening, during its usage. As can be understood, when further air freshening is required, additional sheets of the laminated layers may be pulled open or removed, for dissemination of their fragrance or perfume, into the atmosphere, to sustain the air freshening capabilities of the scenting device of this invention.

Hence, the concept is to provide for a prolonged release of air fresheners, into the ambient air, during the sustained usage of the device of this invention.

In the multi-chamber scenting device, as initially reviewed, each chamber can be opened, selectively, one at a time, or all at once, or two at a time, or any other combination of opening of the device, depending upon the degree and concentration of air freshener that needs to be released, under the circumstances of its usage. Also, each chamber can be reclosed, when air freshening has attained its effective usage, at the option of the user. The chambers of the device can hold all or one fragrance material, or a variety of fragrance materials, thus allowing the consumer to modulate the strength and degree of dissemination from a variety of scented mix, from the device during usage. This allows the device to obtain longevity and variety of usage, whereas most currently available air fresheners usually release only one scent, are usually very strong at the beginning, dissipate rapidly, and cannot be turned down or off during their usage and application. And, such prior art fresheners then fade often within a few days of usage, even if the user is not benefitting from the fragrance, when not within the vicinity of its usage. The current device will allow a user much more firm control over the emanation and freeing of its fragrance, during its usage.

As reviewed, the device of this invention can be constructed from paper, paper lamination as with the third type of embodiment, and with various polymer film layers, as with the first embodiment, and its body or bases can be formed of thermal formed plastics, pressed metal, or be injection molded polymer, in preparing the body member for this device. In the preferred embodiments, the body will be formed of injection molded plastics. But, in the third and more laminated type of air freshener, the body may be formed of a paper, cardboard, or paperboard, or from one of the materials as just described, and then add the laminated layers of film, whether it be polymer or paper, bearing the scented material, laminated onto the formed body, in preparation for its usage and application.

The appearance of these devices can be colored, opaque or clear, or transparent. The preferred embodiment is a transparent plastic. The pull or lift tabs associated with each of the seals, can be made of the same material as the device container, or the tabs may simply be the lateral ends of the seals, that allows for their gripping and removal, in preparation for usage. Or, the tabs may be of a different material, including but not limited to plastic, metal, paper, film structures, including polyester, polyethylene, polypropylene, metabolized films and foils, or combinations thereof. The preferred embodiment is a film structure incorporating polyester.

The lift or pull tabs can be hinged on their ends or unhinged for complete removal. The lift or pull tabs can be decorated by printing, embossing, stamping, perforating, and the like. The separation means between the various seals, as in the preferred embodiment, where there are four seals vertically arranged upon the refreshener body, these seals may be totally separated, or separated by a line of perforations, that allows for the individual removal of each seal, by pulling its pull tab, in preparation for application of the air freshener. The lift tab can be transparent, opaque, or printed with windows that showcase the fragrance material applied therein.

The preferred embodiment would be a printed die-cut film overlay with transparent windows that do show the casing of the body and the fragrance material or gel held therein. The preferred embodiment for the tab attachment would be a repositionable adhesive in the hinge, thus allowing the user to open, close, or pull off the seal completely. Other attachments could include permanent adhesives, chemical bonding, cohesive seals, sonic welded bonds, heat sealing, or a variety of mechanical attachments such as hinges built into the molded device, forming the body with its chambers, and all attachments can be heat sealed or otherwise adhesively applied in place. The opposing side of the scenting device, which can be its front, may also be decorated with branding, graphics, advertisements, or the like. The preferred embodiment would include a printed decorative label, on its front.

The fragrance material can be any fragrance bearing material, including ceramic, paper, felt, scented plastic, non-woven materials, scented gel matrices, or liquid and semi-liquid fragrance formulations, that are enclosed by use of a permanently attached membrane covered by the removable or liftable seals, and their tabs, when it is desired to open and allow for dissemination of the scenting device. The membrane could also be constructed such that the aroma can be migrated through the membrane while containing the fragrance material inside. Membranes like this typically are constructed partially or exclusively with polyethylene. The preferred embodiment is to use a fragranced, colored gel matrix, in application. Examples of such gels are existing types of patented gels, as owned by the Assignee herein, and such can be seen in its U.S. Pat. No. 7,700,665, and in the patent pending published application Ser. No. 13/135,376. These are incorporated herein by reference.

The fragrance delivering device can be suspended by use of an attachment device. The attachment device can be made of paper, metal, wood, plastic, or string, as known in the art. The preferred embodiment is an elastic string, that allows its ease of application, when used in a vehicle, using the rearview mirror to attain its support.

The number of chambers formed into the body of this device can vary from one, to four, as shown in the patent drawings, or it may even be of a longer embodiment, that could have as many as twenty chambers molded into its structure, to hold the fragrance gel(s), and to allow for individual releasing of its seals, to furnish a long term dissemination of its fragrance material to the ambient air, during its prolonged usage. In the preferred embodiment, as noted herein, there are four chambers formed of the freshener body.

The active fragrance material providing the fill or dosage for each chamber can be modified, and can range from 0.2 grams to 100 grams of such gel, in total. The preferred embodiment is 4 to 10 grams total, with 1 or 2.5 grams per chamber. Obviously, the gel will have enough viscosity, so as to prevent its outflow from the formed chamber, in which it locates, once the seal has been removed, so as to allow the gel to dissipate over time, by evaporation, but not to flow freely from any chamber, during its usage and application. The active fragrance material load in the gel would typically range from 50% to 98% of the fragrance material, by weight. The preferred embodiment would be in the vicinity of a 76%, more or less, fragrance load. Another embodiment may include a gel formulation that reduces in size, thus providing an end of life indicator, that can be noted by the user, to determine when it is time to open another seal, or remove and dispose of the fragrance delivering device.

The principal object of this invention is to provide a multi-chambered or laminated layered fragrance disseminating device that provides the means for the consecutive or sequential release of air fragrance either continuously or in staggered periods to furnish a long term release of an air freshener into the ambient atmosphere.

Another object of this invention is to provide the means for furnishing the stepped or timed release of fragrance from a scenting device, to furnish a sequential emanation of the fragrance into the atmosphere, for a much greater length of time than heretofore provided.

Still another object of this invention is to provide a scenting device that can either be fully opened, partially opened, or sequentially opened, so as to control the strength of air freshener being released into the atmosphere, during its usage.

Another object of this invention is to provide a means for variable flow of an air freshener, into the surrounding area.

Still another object of this invention is to provide a scenting device that can be gradually opened, to initiate release of its air freshener, and then be further opened, to continue air freshening, until the device has been fully opened and depleted its contained fragrance material, as supported within its one or more chambered tray.

Still another object of this invention is to provide for a scenting device that includes a body, and a series of fragrance containing laminations, that can be individually pulled free, to release their aroma, perfume, or air freshening scent into the surrounding air.

Another object of this invention is to provide a comprehensive air freshening device that can provide scenting of the surrounding atmosphere for a substantially prolonged period of time.

Another object of this invention is to provide for a unitary air freshening device where its sustained usage can be made through the manipulation of its integral components that furnish either in consecutive or sequential release of an air freshener or fragrance to the atmosphere.

Another object of this invention is to provide a novel air freshener that can be readily employed within a vehicle.

Still another object of this invention is to provide an air freshener that can be used, over a prolonged period of time, in the home or office.

These and other objects may become more apparent to those skilled in the art upon review of the summary of the invention as provided herein, and upon undertaking a study of the description of its preferred embodiments, in view of the drawings.

### BRIEF DESCRIPTION OF THE DRAWINGS

In referring to the drawings:

FIG. 1 is a back view of the multi-chambered and extended release fragrance and scenting device of this invention;

FIG. 2 is a front view thereof;

FIG. 3 is a right side view of the device of FIG. 1;

FIG. 4 is a back view of the device of FIG. 1, with its uppermost polymer or paper firm layer removed to discharge its scent;

FIG. 5 is a vertical sectional view taken along the line 5-5 of FIG. 1;

FIG. 6 shows a multi-layered laminated air freshener of this invention;

FIG. 7 shows the various layers of the laminated air freshener of FIG. 6, capable of selective peeling over periods of time to disseminate their fragrance;

FIG. 8 shows a hanging adjustable slider scenting device of this invention;

FIG. 8a-8d show the sliding cover for the scenting device of FIG. 8 as it is gradually opened to disseminate its fragrance over a sustained period of time; and

FIG. 9 provides a side view of the fragrance slider device of FIG. 8.

### DESCRIPTION OF THE PREFERRED EMBODIMENTS

As previously summarized, the basic concept of this invention is to provide an air freshener, one that can be utilized preferably sequentially, allowing it to freshen the ambient air where it is used, periodically, so as to sustain the scenting or refreshing of the atmosphere in a room, office, vehicle, and for a prolonged period of time. Generally, in the use of the current invention, a certain segment of its air freshener can be opened, to release the aroma from its scented gel, and allow such to be depleted, over a period of time, through this initial usage of the device. Then, when the user deems it necessary to continue the air freshening process, a second section of the same air freshener can be opened, to allow for dissemination of its freshening material, to the atmosphere, to continue the cleansing of the air through usage of this singular device. The device may have a plurality of such segmented freshening components, or many such segments, even as high as twenty or more, to allow for the continuous sequential usage of the device, over a prolonged period of time. Or, if the air freshening requires a significant strength of the scented material to be disseminated into the atmosphere, then a couple of sections of the refreshing device can be opened simultaneously, to allow for a double or more strength of the air freshener to be released into the ambient air, to attain its beneficial results.

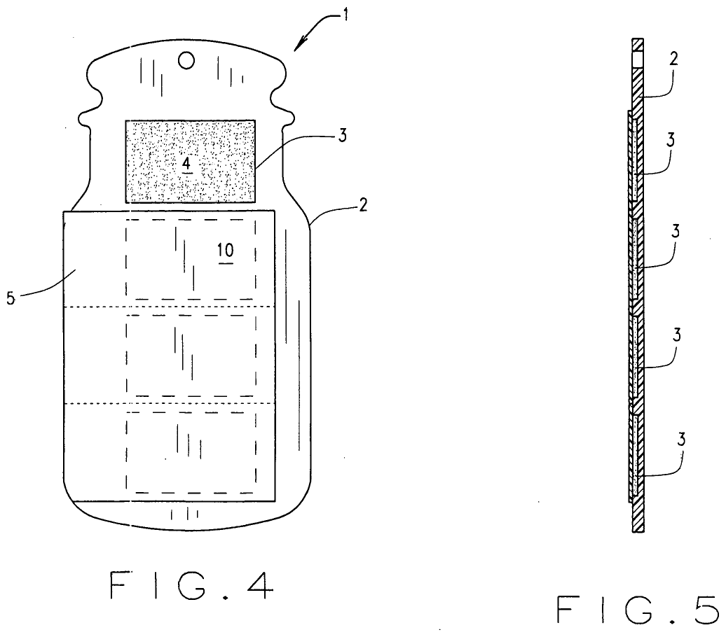

In referring to FIG. 1, this shows one embodiment of the sectionalized air freshener 1, which includes a body portion 2, having a series of a plurality of chambers 3, which form a space within the body 2, for holding a fragrance or air freshening material, usually in gel form, within each of the formed compartments, as can also be noted in FIG. 5. The body 2, and its various chambers 3, can be formed of paper, paperboard, molded of plastic, formed of metal, or even wood, to function as the primary body portion 2 for this freshening device. If it is formed of paperboard, the chambers 3 can be constructed by pressure indentation, or pressed therein, during the formation of the body portion 2. Nevertheless, as noted, it forms a series of chambers, as at 3, for holding the fragrance material, as noted at 4, in FIG. 4. The type of gel as located within each chamber has already been previously reviewed, but generally, is a fragrance type of gel, or a fragrance that may freshen the surrounding air, when used, and which gel has enough viscosity so as to be retained within the chamber, even when it is opened, during its usage, and prevent the scented material from migrating out of the same, particularly when the body portion 2 may be suspended vertically during its usage and application. The characteristics of such a scented gel has already been reviewed herein.

As can be seen in the various figures, the body 2 has an aperture, as at 4, at its upper end, so that a string, cord, clip, or other suspension means may be applied thereto, to allow for the suspension of the device during its usage.

As can also be noted in FIG. 1, there are a series of seal means or strips 5 that are adhered to the back surface 6 of the device, and these seal means are formed as strips of sealing material, whether it be paper, a polymer film such as polyethylene, or other type of sealing material that may be adhesively applied or heat sealed around the perimeter of each chamber 3, in order to seal the various chambers containing their scenting gel in place, as during shipment and storage, or while being displayed for sale, and until such time as the device is readied for usage. Each of these seals, as can be noted, extend laterally, to their edges 7, and which form a tab like portion for each seal, and these tabs may be grasped and pulled free from the device, individually, for purposes as previously explained, to allow for dissemination of its contained scented gel 4 from its exposed chamber, during usage. Generally, the tabs 7 are just not adhered to the back surface 6 of the body 2, so that they can be easily grasped, and pulled free, when opening a particular gel laden chamber of the device, during its usage. As can further be noted, in the preferred embodiment of FIG. 1, each of the seal portions 5 are separated from each other by a line of perforations, as noted at 8, so that, for example, when the top seal member 5 is to be pulled open, it will tear along its line of perforation 8, from the next adjacent seal 5, as can be readily understood. Or, the various seals may simply have, instead of a line of perforation 8, be separated by a cut line from each of the other seal members, so that individual seals 5 can be removed, when it is desired to freshen the surrounding air, by exposing its scented gel to the atmosphere, during usage.

It might also be noted that these seal members 5 can be formed either of opaque film material, or be transparent, so that the user can readily see that there is a fragrance gel supplied within the device, and readied for usage and application, when its seal member is pulled free from the device.

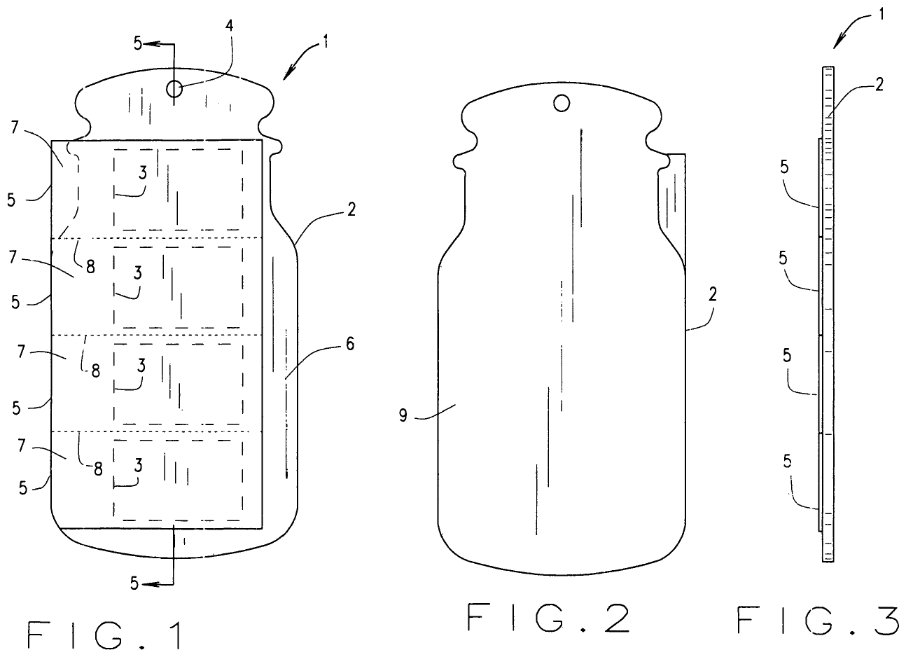

As can also be seen in FIG. 2, the frontal surface 9 extends the full height and width of the body portion 2 of this device, and print indicia may be applied thereto, in the form of directions information, ingredients of its scented material, or even have brand names, trademarks, or other advertising and promotional material applied thereto. Obviously, when this refreshing device is suspended from the rearview mirror of a vehicle, such information can be readily observed by the vehicle occupants.

FIG. 3 shows a side view of the air freshening device, and noting that it is of a thin line construction, and it is not an obstruction to the driver's line of vision, particularly when driving a vehicle.

As noted, in FIG. 4, the upper scented gel holding chamber 3 has had its seal member removed, which exposes its retained gel to the atmosphere, to allow for it to disseminate its refreshing scent to the ambient air, during its usage. For example, it may have been predetermined that through the formulation of the gel 4 supplied into each chamber, in order to control the amount and degree of its discharge of refreshing material into the atmosphere, it may be that one section of the device may provide a week long air freshening, during its usage. Then, when it is necessary to continue the delivery of scented material into the air, a second of the seal member 5 may be removed, to allow for its gel laden container 10 to deliver its scent into the atmosphere, to attain, for example, another week of air freshening through usage of this device 1, as can be understood upon reviewing, once again, the FIG. 4 embodiment.

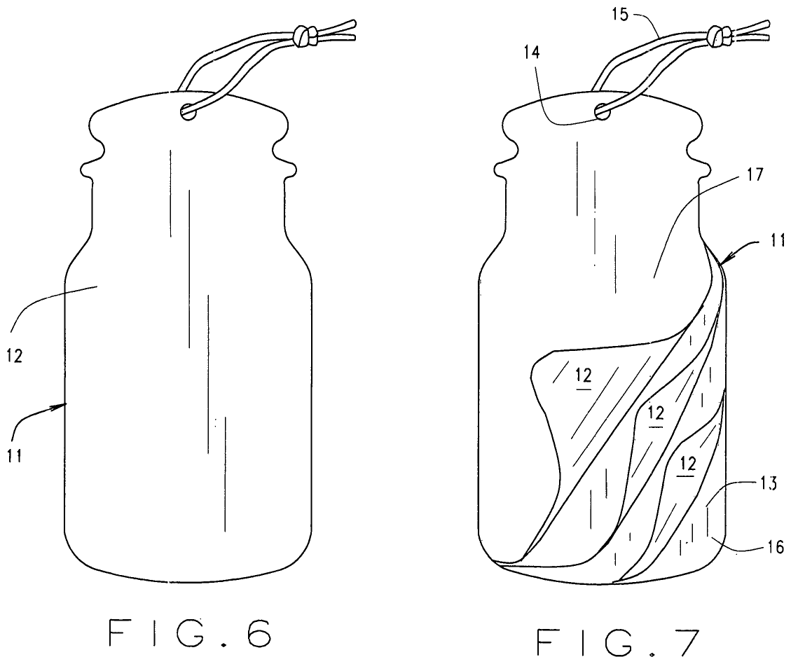

A further example of another embodiment of the air freshener device of this invention can be seen in FIGS. 6 and 7. As noted therein, the device 11 includes a series of laminated layers, as at 12, that are applied to the base material 13 of the formed device. As can also be noted, there is an aperture provided, as at 14, for holding a cord, as at 15, that allows the device to be suspended during usage, as can be understood. This is a die cut type of air freshener, and in this embodiment, can include four highly scented layers 12, including the surface layer 16 of the body member 13, of the formed device. In the preferred embodiment, and these will be formed generally of paper, cardboard or paper board, polymer, generally non-woven layers, not too unlike the types of scented devices that are applied to the pages of magazines, as previously reviewed, so that when each scented layer 12 is removed, its aroma will permeate into the surrounding air, to function as an air freshener, as can be understood. As noted, when the top layer is pulled free, and due to the chemical makeup of its scented material, it may furnish a full week of air freshening, from the suspended device 11. Then, when the second layer is removed, it may provide a second full week of air freshening, from the useful device. And, as noted, there may be a third week and a fourth week that air freshening can be attained, through the peel back of each of its laminate layers, during the usage and application of the shown device. Generally, as can be understood, through the principal of this invention, it is designed to furnish the continuous and consecutive usage of the device, to furnish many weeks of air freshening, from its singular formed structure, as can be understood. One may simply tear the top layer off of the device, either fully separating it, or tearing it at its line perforation, as noted at 17, to expose and activate the next layer of fragrance, constructed into the device. Each of the four layers, in the preferred embodiment, will have had its chemical makeup formulated to last approximately one week of air freshening attributes, and thereby provide a total of four weeks of scenting of the surrounding air. Obviously, it depends upon the chemical makeup of the scented material, and it further depends upon the number of laminations 12 that are constructed into the device during its manufacture, that dictates the longevity of usage of the embodiment, during its application.

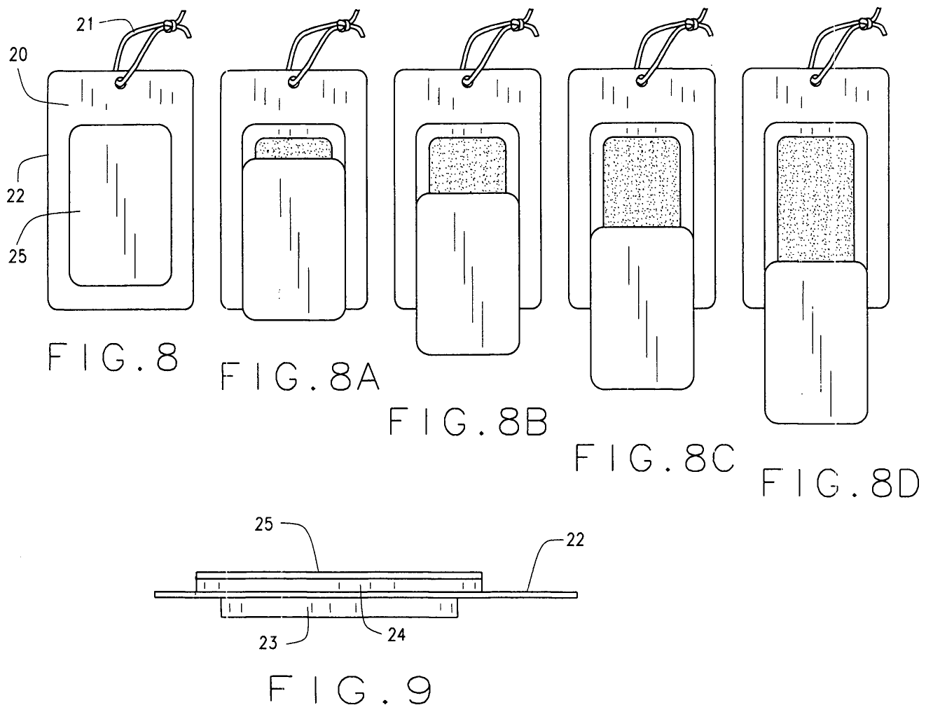

A third embodiment of the concept of this invention can be seen in FIGS. 8, and 8a-8d, in addition to its accompanying FIG. 9. As can be noted, as shown in FIG. 8, the air freshening device 20, during usage, also can be suspended through the application of its cord 21. The device includes a body portion 22 and as can be seen in FIG. 9, it may be formed of a metal or plastic molded casing, formed having its body portion 22, and an integrally formed chamber part 23, as noted. The device may be formed of an aluminum or plastic injection molded casing, that could be mounted in a printed paper card, such as the body or card 22, as noted, so that the entire assembly of the shown device 20 may simply be its card 22, having an aperture therethrough, as noted approximately at 24, wherein the casing including its chamber 23, and its associated lid 25, may be pressed within its body 22, or adhesively secured therein, during its manufacture.

The chamber portion 23 can be filled with a fragrance gel, or scented porous plastic material, which is capable of giving off an aroma, perhaps of perfume, or of an air freshening scent, when it is opened for usage and application. The cover or lid 25 is an adjustable lid, as can be noted in the FIGS. 8a-8d, and can be opened to various positions, for the four positions as noted within said figures, to provide for a weekly freshening for the surrounding air, during the usage and application of the device, as an air freshener. As can be seen, when the lid is shifted downwardly approximately ¼ of the chamber 23, it will provide a calculated one week of air freshening. When the lid is slid approximately ½ downwardly upon its chamber 23, it will provide a second week of air freshening. As can be noted, when pulled further downwardly, it will furnish a week three and a week four of air freshening, until its gel has evaporated completely into the atmosphere, and has been depleted, in its usage for air freshening purposes.

Generally, the device as shown in FIG. 8, is identified as a hanging adjustable slider type of air freshener, in this particular instance, for use generally within an automobile, or at other locations where air freshening is required.

Variations or modifications to the subject matter of this invention may occur to those skilled in the art upon reviewing the summary and structure of the air freshening device as shown and explained herein. Such variations, if within the spirit of this invention, are intended to be encompassed within the scope of any claims to patent protection issuing hereon. The description of the preferred embodiment of this invention, and as depicted in the drawings, are generally set forth for illustrative purposes only.

We claim:

1. An extended usage fragrance release and scenting device, comprising:
a base member, said base member being formed of one of paper, cardboard, paperboard, wood, metal, and polymer, said base member having a plurality of chambers formed integrally therein, each chamber having a quantity of fragrance applied within each chamber, said chambers being formed from one of pressure, indentation, or molded therein, of the base member;
each said chamber being separated from each other chamber as formed within said base member;
a seal for sealing each chamber and its fragrance gel therein, and each seal formed as a thin film of material and made of paper sheet or polymer film, each seal being adhesively applied or heat sealed to said base member and secured thereon for sealing the assembled base member chambers; and
each seal having an extending tab to facilitate the pull opening of its respective seal and allowing exposure of the chambered fragrance gel to the atmosphere, whereby each said seal may be simultaneously or consecutively pulled open from its base member to refresh the surrounding ambient air for a prolonged period of time.

2. The fragrance release device of claim 1, wherein said seals are separately pulled open from its respective chamber to obtain longevity in usage and sustained release of the air freshener to the ambient air.

3. The fragrance release device of claim 2, wherein said base member and its formed chambers are molded of a polymer, and said polymer being one of polypropylene and polyethylene.

4. The fragrance release device of claim 3, wherein the seal films are made of polyethylene.

5. The fragrance release device of claim 4, wherein said seal films are made of transparent polymer.

6. The fragrance release device of claim 4, wherein said films are made of opaque polymer.

7. The fragrance release device of claim 1, wherein the fragrance is a fragrance gel, and said fragrance gel is filled in each base member chamber in an amount between about 0.2 grams to 100 grams.

8. The fragrance release device of claim 7, wherein the fragrance gel is filled within each chamber in an amount between about 4 grams to 10 grams.

9. The fragrance release device of claim 1, wherein the extending tab of each seal is located laterally upon the base member to facilitate its grasping and pulling of the associated seal member into an opened condition.

10. The fragrance release device of claim 1, wherein the chambered fragrance is capable of releasing its scent for up to approximately one week, as each seal is sequentially removed from the device during usage.

11. The fragrance release device of claim 10, and wherein there are four chambers provided in each base member, and when each chambered seal is removed from its base member, it provides for the dissemination of its scent for approximately one week.

12. The fragrance release device of claim 1, wherein each seal is separated by a line of perforations from each adjacent seal in order to facilitate the individual removal of each seal from the base member during usage.

13. The fragrance release device of claim 1, wherein each seal is separated by a line of cut from each adjacent seal in order to facilitate the removal of each individual seal from the base member during application of said device.

14. A long term usage fragrance release and scenting device, comprising:
a base member, said base member being formed of one of paper, cardboard, paperboard, wood, metal or polymer sheet material, said base member having at least one opening provided therein and through said sheet material, said chamber being formed by indentation or molded within said base member within said opening;
a cover for said at least one chamber, said cover slidingly engaged with said base member and its formed chamber and capable of being slid from a closed to an opening position with respect to the formed chamber to gradually open said cover upon the base member chamber to the atmosphere;
said at least one chamber being filled with a quantity of a fragrant or fragranced gel applied therein, whereby when said cover is gradually pulled upon said base member and the associated chamber is gradually opened, the fragrant or fragranced gel is released into the surrounding atmosphere for an extended period of time.

15. The fragrance release device of claim 14, wherein said base member and its formed at least one chamber are molded from a polymer, and said polymer is one of polypropylene and polyethylene.

16. The fragrance release device of claim 14, wherein said base member is formed having an opening provided therethrough, and said formed chamber and slideable cover are pressure fitted within said base member opening, such that when said cover is gradually slid open from the chamber, the fragrance is gradually released into the surrounding atmosphere for an extended period of time.

17. The fragrance release device of claim 16, wherein said cover is made of transparent polymer.

18. The fragrance release device of claim 16, wherein said cover is made of opaque polymer.

19. The fragrance release device of claim 14, wherein said fragrance and fragrance gel is filled in the base member chamber in an amount between about 0.2 grams to 100 grams.

20. The fragrance release device of claim 19, wherein the fragrance gel and fragrance is provided within the formed chamber in an amount between about 4 grams to 10 grams.

21. The fragrance release device of claim 14, wherein the fragrance and fragrance gel is capable of releasing its scent as the cover is gradually slidedly removed from the chamber for up to approximately four weeks.

22. The fragrance release device of claim 14, wherein said cover is formed at its lateral edges with a slide configuration for engagement with the side edges of the formed chamber to allow for the cover to be gradually slid open upon the base member to provide for the gradual release of its scent to the surrounding atmosphere.

23. A multi-layered extended application of a fragrance release and scenting device, comprising:
a base member, said base member being formed of one of paper, cardboard, paperboard, wood, metal, or polymer sheet material, a plurality of laminated sheets applied onto the surface of the base member, each laminated sheet having a quantity of fragrance material applied to their undersurface when laminated together, such that when each sheet is pulled free from the sheet provided therebelow, a fragrance is released to the atmosphere, such that upon removal of each of the laminated sheets from the base member, a timed release of fragrance will be discharged into the surrounding atmosphere, for a sustained period of time.

24. The fragrance release device of claim 23, wherein there are four laminated sheets with the applied scented material laminated onto the base member and which when individually removed, over a period of time, furnishing a discharge of scented refreshener into the atmosphere for approximately four weeks.

25. The fragrance release device of claim 24, wherein said base member includes an aperture at its upper edge, and said aperture capable of holding a cord for suspension of the device during application.
